(19) United States
(12) Patent Application Publication
(10) Pub. No.: US 2026/0125419 A1
(43) Pub. Date: May 7, 2026

(54) PURIFICATION METHODS

(71) Applicant: The New Zealand Institute for Plant and Food Research Limited, Auckland (NZ)

(72) Inventor: Mathew Hoani CUMMING, Audkland (NZ)

(21) Appl. No.: 19/118,282

(22) PCT Filed: Oct. 4, 2023
(86) PCT No.: PCT/IB2023/059942

§ 371 Date: Apr. 3, 2025

(30) Foreign Application Priority Data
Oct. 4, 2022 (AU) 2022902880

(51) Int. Cl. C07K 1/14; A61K 38/39; C07K 14/78

(52) U.S. Cl. CPC C07K 1/145; A61K 38/39; C07K 14/78

(57) ABSTRACT

This invention relates to methods for purifying type II collagen from cartilage tissue. The present invention also relates to a type II collagen composition, and the use of the type II collagen composition as a dietary supplement or treatment for cartilage-related disorders. Methods of treating or preventing cartilage-related disorders are also provided.

### FIELD OF THE INVENTION

The present invention relates to methods for purifying type II collagen from cartilage tissue. The present invention also relates to a type II collagen composition, and the use of the type II collagen composition as a dietary supplement or treatment for cartilage-related disorders.

### BACKGROUND OF THE INVENTION

Type II collagen has shown promising beneficial effects on human health such as improving joint function and reducing joint inflammation (Harris et al., J. Diet Suppl. 2021, 1:1-16). It has also shown promise as a treatment for cartilage-related disorders, such as osteoarthrosis and rheumatoid arthritis (Scarpellini et al., J Orthop Traumatol. 2008; 9(2): 81-7, and Crowley et al., Int J Med Sci. 2009; 9; 6(6): 312-21). The beneficial effects of type II collagen are believed to result from the binding of tolerising epitopes present in undenatured type II collagen to Peyer's patches in the intestine. Through these interactions, the type II collagen is believed to elicit an immune response that reduces joint inflammation and discomfort (Harris et al., J. Diet Suppl. 2021, 1:1-16).

Examples of type II collagen products that are commercially available and reported to contain undenatured type II collagen include Collavant n2® (previously known as b-2Cool®, Bioberica, S.A.U.; Barcelona, Spain) and UC-II® (Lonza Consumer Health Inc, USA). Both products are reported to be derived from chicken sternal cartilage.

Common methods for obtaining type II collagen from cartilage tissue involve washing, drying and grinding cartilage tissue followed by treatment with guanidinium hydrochloride and/or enzymatic digestion (for example with trypsin, pepsin and/or elastase) to remove proteoglycans and glycosaminoglycans, and solubilise type II collagen. The cartilage tissue is typically obtained from livestock animals or poultry. Treatment of cartilage tissue with guanidinium hydrochloride and/or enzymatic digestion can lead to denaturation and/or hydrolysis of the collagen in the cartilage. As a result, type II collagen products prepared by such methods often contain low levels of undenatured type II collagen.

Thus, there remains a need for methods suitable for obtaining type II collagen, in particular methods for obtaining undenatured type II collagen suitable for use in pharmaceuticals and dietary supplements. It is also an object of the present invention to provide an improved or alternative method or composition, and/or to at least provide the public with a useful choice.

### SUMMARY OF THE INVENTION

The present invention provides a method for purifying type II collagen from cartilage tissue, said method comprising:
i) providing a sample of cartilage tissue;
ii) contacting the sample with a salt solution under conditions that allow at least one component of the cartilage tissue to dissolve in the salt solution, wherein the at least one component of the cartilage tissue is selected from the group consisting of proteoglycan, glycosaminoglycan, and calcium salt (e.g. calcium phosphate); and
iii) removing the salt solution and the at least one component of the cartilage tissue from the sample;
wherein step iii) removes at least about 25% of the glycosaminoglycan from the sample; and wherein, following step iii), the sample comprises type II collagen of which at least about 50% (w/w) is undenatured type II collagen.

The sample provided following step iii) of the method of the invention is referred to herein as a type II collagen composition. The present inventors have found that the type II collagen composition obtained from the method of the invention comprises undenatured type II collagen at a level and purity that make it particularly suitable for use as a medicament or dietary supplement.

The present inventors have also found that the method of the invention is particularly effective at purifying undenatured type II collagen from fish cartilage, and particularly fish cartilage from fish parts generally discarded during commercial processing of fish for consumption. This feature of the present invention enables the production of low-cost undenatured type II collagen products with a limited impact on the environment.

For the avoidance of doubt, the term “undenatured type II collagen” as used herein refers to type II collagen that retains its native triple helix structure. This contrasts with denatured type II collagen, which has lost its native triple helix structure. An example of denatured type II collagen is type II collagen that has been hydrolyzed into peptide fragments (often referred to as collagen hydrolysates). Undenatured type II collagen typically has a molecular weight of about 300 kDa, whereas the molecular weight of collagen hydrolysates ranges from about 2 kDa to about 9 kDa. Typically, undenatured type II collagen is insoluble in aqueous solutions whereas denatured type II collagen is soluble in aqueous solutions. The term “type II collagen” as used herein is to be understood as referring to undenatured type II collagen, denatured type II collagen, or a mixture thereof, unless otherwise stated.

The present invention also provides a type II collagen composition obtained by the method of the present invention. The type II collagen composition of the invention finds use as a medicament, such as a medicament for the treatment or prevention of cartilage-related disorders. The type II collagen composition of the invention also finds use as a dietary supplement, for example as a dietary supplement to promote joint health.

The present invention further provides methods of treating cartilage-related disorders, and a type II collagen composition for use in the manufacture of a medicament for the treatment of cartilage-related disorders.

### DESCRIPTION OF THE DRAWINGS

FIG. 1 shows the amount of glycosaminoglycans (GAGs) extracted from cartilage tissue from hoki nose cartilage or chicken sternum under various conditions. The conditions shown on the x-axis that include “CS” were used with chicken sternum cartilage. Condition shown on the x-axis that do not include “CS”, or include “HN”, were used with hoki nose cartilage. In FIG. 1: AcAc/AcOH=acetic acid; TBP=tributyl phosphate; chaps=(3-((3-cholamidopropyl) dimethylammonio)-1-propanesulfonate); and GuHCl=guanidinium hydrochloride.

FIG. 2 shows the amounts of GAGs and hydroxyproline extracted from cartilage tissue from hoki nose cartilage or chicken sternum under various conditions. In FIG. 2: HN=hoki nose cartilage; CS=Chicken sternum cartilage; AcOH=acetic acid; and GuHCl=guanidinium hydrochloride.

FIG. 3 shows the amount of GAGs extracted from cartilage tissue from dogfish spine (DF), hoki nose cartilage (HN) and chicken sternum (CS) using a sodium sulfate solution.

FIG. 4 shows the total hydroxyproline in cartilage tissue samples obtained from dogfish spine (DF), hoki nose cartilage (HN) and chicken sternum (CS). The processed cartilage tissue samples (DF, HN and CS processed) were treated with a sodium sulfate solution before analysis.

FIG. 5 shows the percentage of the collagen in cartilage tissue samples that is undenatured collagen. The cartilage tissue analysed in this experiment was obtained from dogfish spine (DF), hoki nose cartilage (HN) and chicken sternum (CS). The processed cartilage tissue samples (DF, HN and CS processed) were treated with a sodium sulfate solution before analysis.

FIG. 6 shows GAG extraction from (A) chicken sternum and (B) hoki nose cartilage tissue using 0.5 M sulfate salt and chloride salt solutions with a pH 4, pH 7 and pH 9. * indicates precipitation of material following addition of the salt solution to the processed cartilage tissue sample.

FIG. 7 shows GAG extraction from (A) Chicken Sternum and (B) Hoki nose cartilage tissue using 1.0 M sulfate salt and chloride salt solutions with a pH 4, pH 7 and pH 9. * indicates precipitation of material following addition of the salt solution to the processed cartilage tissue sample.

FIG. 8 shows the percentage of undenatured collagen based on the percentage of hydroxyproline in the pellet formed following treatment of Pellet A samples with trypsin. The ratio of hydroxyproline in the pellet is an indirect measure of undenatured collagen content in the cartilage tissue sample following treatment with an extraction reagent. The Pellet A samples were generated by treating hoki nose cartilage tissue samples with 0.5 M sodium sulfate solutions with a pH of about 4, 7 or 9, or 1 M magnesium sulfate solutions with a pH of about 4, 7 or 9. The amounts of GAGs extracted by each solution from cartilage tissue samples shown in FIGS. 6 and 7 are overlayed in FIG. 8 and shown as “x”.

FIG. 9 shows scanning electron micrographs of collagen produced using 1M MgSO₄ or pepsin treatment. (A) MgSO₄ treatment, 534× magnification. (B) pepsin treatment, 534× magnification. (C) MgSO₄ treatment, 133,000× magnification. (D) pepsin treatment, 133,000× magnification.

### DETAILED DESCRIPTION OF THE INVENTION

As described herein, the present inventors have developed a method for purifying type II collagen, in particular undenatured type II collagen, from cartilage tissue. The method of the invention is performed in vitro. As set out above, the method comprises the steps of:
i) providing a sample of cartilage tissue;
ii) contacting the sample with a salt solution under conditions that allow at least one component of the cartilage tissue to dissolve in the salt solution, wherein the at least one component is selected from the group consisting of proteoglycan, glycosaminoglycan, and calcium salt (e.g. calcium phosphate); and
iii) removing the salt solution and the at least one dissolved component from the sample; wherein step iii) removes at least about 25% of the glycosaminoglycan from the sample; and wherein, following step iii), the sample comprises type II collagen of which at least about 50% (w/w) is undenatured type II collagen.

Also provided herein is a method for purifying type II collagen from cartilage tissue, said method comprising:
i) providing a sample of cartilage tissue from a bony fish in the Merlucciidae family (for example, a blue grenadier, also known as Macruronus novaezelandiae and hoki), a cartilaginous fish (for example, a dogfish, blue shark, and mako) or a bird (for example, poultry such as chicken, turkey, duck, goose, guinea fowl, and pigeon);
ii) contacting the sample with a chloride salt solution or sulfate salt solution under conditions that allow at least one component of the cartilage tissue to dissolve in the salt solution, wherein the at least one component of the cartilage tissue is selected from the group consisting of proteoglycan, glycosaminoglycan, and calcium salt (e.g. calcium phosphate); and
iii) removing the salt solution and the at least one component of the cartilage tissue from the sample.

For the avoidance of doubt, the term “comprising” as used herein means “consisting at least in part of”. When interpreting statements in this specification which include that term, the features, prefaced by that term in each statement or claim, all need to be present but other features can also be present. The related terms “comprises” and “comprised” are to be interpreted similarly. In addition, as used herein, the term “and/or” means “and” or “or”, or both. Also, for the avoidance of doubt, the term “purifying type II collagen from cartilage tissue” as used herein may be understood as a process of removing a proteoglycan, glycosaminoglycan, and/or calcium salt (e.g. calcium phosphate) from a cartilage tissue sample. The proteoglycan, glycosaminoglycan, and/or calcium salt (e.g. calcium phosphate) are removed from the cartilage tissue sample in step iii) of the method of the invention.

Typically, the method of the invention removes at least about 0.1%, 0.2%, 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.8%, 99.9% or 100% of the proteoglycans, glycosaminoglycans, and/or calcium salt (e.g. calcium phosphate) from the cartilage tissue sample. For example, the method of the invention may remove about 0.1% to about 50%, about 0.2% to about 50%, about 0.5% to about 50%, about 1% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 60%, about 0.2% to about 60%, about 0.5% to about 60%, about 1% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 0.1% to about 70%, about 0.2% to about 70%, about 0.5% to about 70%, about 1% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 0.1% to about 80%, about 0.2% to about 80%, about 0.5% to about 80%, about 1% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 0.1% to about 90%, about 0.2% to about 90%, about 0.5% to about 90%, about 1% to about 90%, about 5% to about 90%, about 10% to about 90%, about 15% to about 90%, about 20% to about 90%, about 25% to about 90%, about 30% to about 90%, about 35% to about 90%, about 40% to about 90%, about 45% to about 90%, about 0.1% to about 99%, about 0.2% to about 99%, about 0.5% to about 99%, about 1% to about 99%, about 5% to about 99%, about 10% to about 99%, about 15% to about 99%, about 20% to about 99%, about 25% to about 99%, about 30% to about 99%, about 35% to about 99%, about 40% to about 99%, and about 45% to about 99%, about 0.1% to about 100%, about 0.2% to about 100%, about 0.5% to about 100%, about 1% to about 100%, about 5% to about 100%, about 10% to about 100%, about 15% to about 100%, about 20% to about 100%, about 25% to about 100%, about 30% to about 100%, about 35% to about 100%, about 40% to about 100%, and about 45% to about 100% of the proteoglycans, glycosaminoglycans, and/or calcium salt (e.g. calcium phosphate) from the cartilage tissue sample.

Typically, the calcium salt removed from the cartilage tissue is calcium phosphate. Preferably, the method of the invention removes about 10% to about 100% of the proteoglycans, glycosaminoglycans, and/or calcium salt (e.g. calcium phosphate) from the cartilage tissue sample. For example, a cartilage tissue sample that comprises about 100 mg of proteoglycans before being subjected to the method of the invention may contain about 0 mg to about 90 mg of proteoglycans following step iii) of the method. Or, for example, a cartilage tissue sample that comprises about 100 mg of glycosaminoglycans before being subjected to the method of the invention may contain about 0 mg to about 90 mg of glycosaminoglycans following step iii) of the method. Or, for example, a cartilage tissue sample that comprises about 100 mg calcium salt (e.g. calcium phosphate) before being subjected to the method of the invention may contain about 0 mg to about 90 mg calcium salt (e.g. calcium phosphate) following step iii) of the method.

Methods for determining the proteoglycan, glycosaminoglycan, and/or calcium salt (e.g. calcium phosphate) content of a cartilage tissue sample are known in the art. For example, proteoglycan and/or glycosaminoglycan can be detected and quantified using dimethylmethylene blue (DMMB) based assay, mass spectrometry, sodium dodecyl sulfate-polyacrylamide (SDS) gel electrophoresis, spectrophotometry and/or colorimetric protein assays such as Coomassie blue based assays. Calcium salts such as calcium phosphate may be detected and quantified using ammonium molybdate and/or Arsenazo III (CAS No. 1668-00-4) based assays, and/or using inductively coupled plasma mass spectrometry (ICP-MS). Other techniques such as ELISA, liquid chromatography mass spectrometry, and liquid chromatography-mass spectrometry (LC-MS) may also be used. Commercial assay kits for detecting and quantifying proteoglycan, glycosaminoglycan, and/or calcium salt (e.g. calcium phosphate) are also widely available. Further suitable methods are also described in the Examples disclosed herein.

The present inventors have found that by removing the proteoglycans, glycosaminoglycans, and/or calcium salt (e.g. calcium phosphate) from a cartilage tissue sample, the percentage (w/w) of type II collagen in the sample is increased. Typically, following step iii) of the method of the invention, the percentage (w/w) of type II collagen in the cartilage tissue sample is at least about 0.1%, 0.2%, 0.5%, 1%, 5%, 10%, 15%, 20%, 30%, 40%, 50%, 60%, 70%, 80%, 90%, 100%, 110%, 120%, 130%, 140%, 150%, 160%, 170%, 180%, 190% or 200% greater than the percentage (w/w) of type II collagen in the cartilage tissue sample before step iii) of the method. Preferably, following step iii) of the method of the invention, the percentage (w/w) of type II collagen in the cartilage tissue sample is at least about 5%, 10%, 15%, 20%, 30%, 40%, 50%, 60%, 70%, 80%, 90% or 100% greater than the percentage (w/w) of type II collagen in the cartilage tissue sample before step iii) of the method, more preferably at least about 40%, 50%, 60%, 70%, 80%, 90% or 100% greater than the percentage (w/w) of type II collagen in the cartilage tissue sample before step iii) of the method. For example, following step iii) of the method of the invention, the percentage (w/w) of type II collagen in the cartilage tissue sample may be about 40% to about 100%, about 45% to about 100%, about 45% to about 90%, about 45% to about 85%, or about 45% to about 80% greater than the percentage (w/w) of type II collagen in the cartilage tissue sample before step iii) method.

Typically, following step iii) of the method of the invention, the percentage (w/w) of type II collagen in the cartilage tissue sample is about 40% to about 100% greater than the percentage (w/w) of type II collagen in the cartilage tissue sample before step iii) method. For example, a cartilage tissue sample that comprises about 10% (w/w) type II collagen before being subjected to the method of the invention may contain about 14% (w/w) to about 20% (w/w) type II collagen following step iii) of the method of the invention. Or, for example, a cartilage tissue sample that comprises about 10 mg type II collagen per 100 mg of cartilage tissue sample before being subjected to the method of the invention may contain about 14 mg to about 20 mg type II collagen per 100 mg of the sample following step iii) of the method.

For the avoidance of doubt, the term “type II collagen” as used herein is to be understood as referring to undenatured type II collagen, denatured type II collagen, or a mixture thereof, unless otherwise stated. Accordingly, the method of the invention may increase the percentage (w/w) of undenatured type II collagen, denatured type II collagen, or a mixture thereof in a cartilage tissue sample by the percentages disclosed hereinabove. Preferably, the method of the invention increases the percentage (w/w) of undenatured type II collagen in a cartilage tissue sample by the percentages disclosed hereinabove. For example, following step iii) of the method of the invention, the percentage (w/w) of undenatured type II collagen in the cartilage tissue sample may be at least about 5%, 10%, 15%, 20%, 30%, 40%, 50%, 60%, 70%, 80%, 90% or 100% greater than the percentage (w/w) of undenatured type II collagen in the cartilage tissue sample before step iii) of the method.

For the avoidance of doubt, the term “w/w” as used herein refers to weight for weight of a particular substance within a mixture. For example, a sample containing 10% (w/w) undenatured type II collagen is to be understood as containing 10 mg of undenatured type II collagen for every 100 mg of the sample. Unless stated otherwise, any reference herein to the mass of type II cartilage, undenatured type II collagen, or cartilage tissue sample is to be understood as referring to the dry mass of the type II cartilage, undenatured type II collagen, or cartilage tissue sample.

Methods for determining the type II collagen content (i.e. the sum of undenatured and denatured type II collagen content) and undenatured type II collagen content of a sample are known in the art. For example, Harris et al., (J. Diet Suppl. 2021, 1:1-16, which is hereby incorporated herein by reference) describe the determination of type II collagen content of a sample by hydrolysing the protein in the sample using hydrochloric acid, and measuring the hydroxyproline content. Harris et al., also describe the determination of undenatured type II collagen content of a sample using an enzyme-linked immunosorbent assay (ELISA) based method that uses a monoclonal antibody specific for undenatured type II collagen. Alternative methods for determining the undenatured type II collagen content of a sample include trypsin digestion susceptibility assays such as those described by Veres et al., and Bank et al., (Matrix Biol, 2014, 33:54-59 and Matrix Biol, 1997, 16(5): 233-243 which are hereby incorporated herein by reference). The type II collagen content and undenatured type II collagen content of a sample may also be determined by measuring the average molecular weight of the collagen in the composition, for example, by using methods known in the art such as liquid chromatography, mass spectrometry, and liquid chromatography-mass spectrometry (LC-MS). Commercial assay kits suitable for determining type II collagen content and undenatured type II collagen content of a sample are widely available. Further suitable methods are also described in the Examples disclosed herein.

### Cartilage Tissue

Cartilage tissue has a matrix structure comprising collagen, chondrocytes, proteoglycans, and glycosaminoglycans (GAGs) such as chondroitin sulfate, heparan sulfate, heparin, dermatan sulfate, keratan sulfate and hyaluronic acid. Typically, cartilage tissue contains about 60-80% water, 10-20% collagen, 20-30% proteoglycans and glycosaminoglycans, and 1-5% chondrocytes. The cartilage tissue of cartilaginous fish (i.e. a Chondrichthyes) such as shark typically also comprises calcium salts (e.g. calcium phosphate). Chondrichthyes cartilage can contain about 15-25% calcium salts.

Type II collagen is typically found in hyaline cartilage tissue, where it functions as a matrix molecule that provides structural integrity. Hyaline cartilage tissue is found on joint surfaces and as a structural component of the rib cage neck and bronchial tubes. In cartilaginous fish, cartilage can constitute a significant proportion of the skeleton. Preferably, the cartilage tissue sample used in the method of the invention is a hyaline cartilage tissue sample. Typically, the cartilage tissue sample used according to the method of the invention is obtained from a fish, bird, mammal, mollusc, or echinoderm. For example, the cartilage tissue sample may be a bony fish (i.e. an Osteichthyes) such as cod, haddock, pollock, hake, tuna, salmon (e.g., Salmo salar), sturgeon, or trout, or a cartilaginous fish such as shark, ray, skate or sawfish. Or, for example, the cartilage tissue sample may be from a bird such as poultry (for example, chicken, turkey, duck, goose, guinea fowl, and pigeon). Or, for example, the cartilage tissue sample may be from a mammal such as a livestock animal (for example, a domestic cow, domestic pig, horse, donkey, zebu, bali cattle, yak, water buffalo, gayal, sheep, reindeer, camel, llama, alpaca, rabbit, and guinea pig). Or, for example, the cartilage tissue sample may be from a mollusc such as a squid, cuttlefish or octopus. Or, for example, the cartilage tissue sample may be from an echinoderm such as a sea cucumber, star fish or sea urchin. Preferably, the cartilage tissue sample is from a fish, bird, or mammal.

When the cartilage tissue sample is from a bony fish, the fish may be one classified in the Gadiformes order, such as a cod, haddock, pollock, or hake (for example, a blue grenadier, also known as Macruronus novaezelandiae or Hoki, or Merluccius merluccius, also known as European hake). Or, for example, the bony fish may be one classified in the Salmoniformes order, such as a trout or a salmon (for example, Salmo salar). Preferably, the bony fish is a blue grenadier.

When the cartilage tissue sample is from a cartilaginous fish, the fish may be a shark. For example, the cartilaginous fish may be dogfish (Squalus acanthias), blue shark (Prionace glauca), or mako shark (e.g. shortfin mako (Isurus oxyrinchus) and longfin mako (Isurus paucus)).

When the cartilage tissue sample is from a bird, the bird may be poultry. For example, the bird may be a chicken or turkey. Preferably, the bird is a chicken.

When the cartilage tissue sample is from a mammal, the mammal may be a livestock animal. For example, the mammal may be a domestic cow.

The present inventors have found bony fish and birds to be particularly suitable sources of cartilage tissue for use according to the present invention. Thus, in certain preferred embodiments, the cartilage tissue sample is from a bony fish or a bird, for example a blue grenadier or a chicken.

The cartilage tissue sample may be obtained from an animal that is discarded as bycatch during animal or plant harvesting or collecting, or the cartilage tissue sample may be obtained from an animal body part that is typically discarded when the animal is processed for consumption. Discarded bycatch and animal parts provide a low-cost source of cartilage suitable for use according to the present invention. Examples of animal parts that may be used as a source of cartilage tissue include fish heads, vertebral columns, fish fins, and sternums (for example, chicken or turkey sternums), or parts thereof. Nasal cartilage from bony fish, cartilage from bird sternums, and cartilage from shark vertebral columns have been found to be particularly suitable for use according to the present invention.

In one exemplary embodiment, the cartilage tissue sample is from a cod, haddock, pollock, salmon (for example, Salmo salar), European hake (also known as a Merluccius merluccius) or blue grenadier (also known as a Macruronus novaezelandiae).

In another exemplary embodiment, the cartilage tissue sample is nasal cartilage tissue from a blue grenadier. In another exemplary embodiment, the cartilage tissue sample is from the vertebral column, or a part thereof, of a shark (for example, a dogfish, blue mako or shortfin mako). In another exemplary embodiment, the cartilage tissue sample is from a chicken sternum.

### Salt Solutions

The salt solution used in the method of the invention may have a pH of about 3 to about 10, such as about 3 to about 9. Salt solutions with a pH of about 3 to about 10 have been found to be effective at dissolving proteoglycan, glycosaminoglycan, and calcium salt (e.g. calcium phosphate) in a cartilage tissue sample whilst maintaining, or increasing, the percentage (w/w) undenatured type II collagen in a sample. Typically, the salt solution has a pH of about 3 to about 10, about 3.5 to about 10, 4 to about 10, about 4.5 to 10, about 5 to about 10, about 5.5 to about 10, about 6 to about 10, about 6.5 to about 10, about 7 to about 10, about 7.5 to about 10, about 8 to about 10, about 8.5 to about 10, about 9 to about 10, about 9.5 to about 10, about 3 to about 9.5, about 3.5 to about 9.5, 4 to about 9.5, about 4.5 to 9.5, about 5 to about 9.5, about 5.5 to about 9.5, about 6 to about 9.5, about 6.5 to about 9.5, about 7 to about 9.5, about 7.5 to about 9.5, about 8 to about 9.5, about 8.5 to about 9.5, about 9 to about 9.5, about 3 to about 9, about 3.5 to about 9, 4 to about 9, about 4.5 to 9, about 5 to about 9, about 5.5 to about 9, about 6 to about 9, about 6.5 to about 9, about 7 to about 9, about 7.5 to about 9, about 8 to about 9, about 3 to about 8.5, about 3.5 to about 8.5, 4 to about 8.5, about 4.5 to 8.5, about 5 to about 8.5, about 5.5 to about 8.5, about 6 to about 8.5, about 6.5 to about 8.5, about 7 to about 8.5, about 7.5 to about 8.5, about 8 to about 8.5, 3 to about 8, about 3.5 to about 8, about 4 to about 8, about 4.5 to 8, about 5 to about 8, about 5.5 to about 8, about 6 to about 8, about 6.5 to about 8, about 7 to about 8, about 7.5 to about 8, about 3 to about 7.5, about 3.5 to about 7.5, about 4 to about 7.5, about 4.5 to 7.5, about 5 to about 7.5, about 5.5 to about 7.5, about 6 to about 7.5, about 6.5 to about 7.5, about 7 to about 7.5, about 3 to about 7, about 3.5 to about 7, about 4 to about 7, about 4.5 to 7, about 5 to about 7, about 5.5 to about 7, about 6 to about 7, about 6.5 to about 7, about 3 to about 6.5, about 3.5 to about 6.5, about 4 to about 6.5, about 4.5 to 6.5, about 5 to about 6.5, about 5.5 to about 6.5, about 6 to about 6.5, about 3 to about 6, about 3.5 to about 6, about 4 to about 6, about 4.5 to 6, about 5 to about 6, about 5.5 to about 6, about 3 to about 5.5, about 3.5 to about 5.5, about 4 to about 5.5, about 4.5 to 5.5, about 4 to about 5, about 4.5 to about 5, about 3 to about 4.5, or about 3.5 to about 4.5.

In certain embodiments, the salt solution has a pH of about 3.5 to about 7, or about 4 to about 7 (for example, a pH of about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.6, about 5.7, about 5.8, about 5.9, about 6, about 6.1, about 6.2, about 6.3, about 6.4, about 6.5, about 6.7, about 6.8, about 6.9, or about 7). For example, the salt solution may have a pH of about 3.5 to about 6, about 3.5 to about 5, about 3.5 to about 4.5, about 4 to about 7, about 4 to about 6, or about 4 to about 5. In certain preferred embodiments, the salt solution has a pH of about 3.5 to about 4.5 (for example, a pH of about 4).

In some embodiments, the salt solution (such as a sodium sulfate salt solution) has a pH of about 3 to about 5 or about 8 to about 10, preferably about 3.5 to about 4.5 or about 8.5 to about 9.5, more preferably about 4 or about 9. In some embodiments, the salt solution (such as a magnesium sulfate salt solution) has a pH of about 4 to about 6 or about 8 to about 10, preferably about 4.5 to about 5.5 or about 9 to about 9.75, more preferably about 5 or about 9. The salt solution may be an inorganic salt solution or an organic salt solution. For example, the salt solution may be a chloride, sulfate or acetate salt solution. Suitable salts include, for example, sodium chloride, potassium chloride, magnesium chloride, calcium chloride, ammonium chloride, sodium sulfate, magnesium sulfate, ammonium sulfate and sodium acetate. Preferably, the salt solution is a chloride salt solution or a sulfate salt solution. For example, the salt solution may be a sodium chloride, potassium chloride, magnesium chloride, calcium chloride, ammonium chloride, sodium sulfate, magnesium sulfate, ammonium sulfate salt solution. The present inventors have found that sodium sulfate solutions and sodium chloride solutions are particularly effective at solubilising proteoglycans, glycosaminoglycans and/or calcium salts (e.g. calcium phosphate) in cartilage tissue. Accordingly, in certain embodiments, the salt solution is a sodium sulfate solution or a sodium chloride solution. Preferably, the salt solution is a sodium sulfate solution.

In some embodiments, the salt is a metal salt (e.g. a sodium salt or a magnesium salt, such as sodium chloride, sodium sulfate, magnesium chloride, or magnesium sulfate) or an ammonium salt (e.g. ammonium chloride or ammonium sulfate). In some embodiments, the salt is a metal salt (e.g. a sodium salt or a magnesium salt, such as sodium chloride, sodium sulfate, magnesium chloride, or magnesium sulfate).

In some embodiments, the salt is a non-chaotropic salt. In some embodiments, the salt solution does not comprise a chaotropic salt. Chaotropic salts are salts that disrupt hydrogen bonds within and/or between proteins, and are therefore capable of denaturing proteins in solution. One commonly used chaotropic salt is guanidinium chloride (also known as guanidine hydrochloride). Non-chaotropic salts are salts which do not possess chaotropic properties. Anti-chaotropic salts (also known as kosmotropic salts) stabilise hydrogen bonds, resulting in increased protein stability.

Typically, the salt solution has a salt concentration of about 0.1 M to about 2 M. For example, about 0.2 M to about 2 M, about 0.3 M to about 2 M, about 0.4 M to about 2 M, about 0.4 M to about 1.75M, 0.5 M to about 1.5M, about 0.75 M to about 1.5 M, 0.75 M to about 1.25 M, about 0.8 M to about 1.2 M, or about 0.9 M to about 1.1 M.

Chloride salt solutions and sulfate salt solutions have been found to be particularly effective at dissolving glycosaminoglycan and calcium salts (e.g. calcium phosphate) in cartilage tissue when the salt is present at a concentration of about 0.4 M to about 2 M. Thus, in certain embodiments, the salt solution is a chloride salt solution or sulfate salt solution with a chloride or sulfate concentration of about 0.4 M to about 2 M. For example, the chloride or sulfate salt may be present at a concentration of about 0.4 M to about 1.75M, 0.5 M to about 1.5 M, about 0.75 M to about 1.5 M, 0.75 M to about 1.25 M, about 0.8 M to about 1.2 M, or about 0.9 M to about 1.1 M. Or, for example, the chloride or sulfate salt may be present at a concentration of about 0.4 M, about 0.5 M, about 0.75 M, about 1 M, about 1.5 M or about 2 M. Salt solutions with a chloride or sulfate concentration of about 1 M have been found to be particularly effective. Accordingly, in certain preferred embodiments, the chloride or sulfate salt is present at a concentration of about 1 M.

In some particularly contemplated embodiments, sodium sulfate may be present at a concentration of at least about 0.2 M, such as at least about 0.3 M, at least about 0.4 M, at least about 0.5 M, at least about 0.6 M, at least about 0.7 M, at least about 0.8 M, at least about 0.9 M, at least about 1 M, at least about 1.1 M, or at least about 1.2 M, and useful ranges may be selected between any of these values (for example, from about 0.2 M to about 1.2 M, from about 0.2 M to about 1.1 M, from about 0.2 M to about 1 M, from about 0.2 M to about 0.9 M, from about 0.2 M to about 0.8 M, from about 0.2 M to about 0.7 M, from about 0.2 M to about 0.6 M, from about 0.3 M to about 1.2 M, from about 0.3 M to about 1.1 M, from about 0.3 M to about 1 M, from about 0.3 M to about 0.9 M, from about 0.3 M to about 0.8 M, from about 0.3 M to about 0.7 M, from about 0.3 M to about 0.6 M, from about 0.4 M to about 1.2 M, from about 0.4 M to about 1.1 M, from about 0.4 M to about 1 M, from about 0.4 M to about 0.9 M, from about 0.4 M to about 0.8 M, from about 0.4 M to about 0.7 M, from about 0.4 M to about 0.6 M), preferably about 0.4 M.

In some particularly contemplated embodiments, magnesium sulfate may be present at a concentration of at least about 0.2 M, such as at least about 0.3 M, at least about 0.4 M, at least about 0.5 M, at least about 0.6 M, at least about 0.7 M, at least about 0.8 M, at least about 0.9 M, at least about 1 M, at least about 1.1 M, at least about 1.2 M, at least about 1.3 M, or at least about 1.4 M, and useful ranges may be selected between any of these values (for example, from about 0.2 M to about 1.4 M, from about 0.2 M to about 1.3 M, from about 0.2 M to about 1.2 M, from about 0.2 M to about 1.1 M, from about 0.2 M to about 1 M, from about 0.3 M to about 1.4 M, from about 0.3 M to about 1.3 M, from about 0.3 M to about 1.2 M, from about 0.3 M to about 1.1 M, from about 0.3 M to about 1 M, from about 0.4 M to about 1.4 M, from about 0.4 M to about 1.3 M, from about 0.4 M to about 1.2 M, from about 0.4 M to about 1.1 M, from about 0.4 M to about 1 M, from about 0.5 M to about 1.4 M, from about 0.5 M to about 1.3 M, from about 0.5 M to about 1.2 M, from about 0.5 M to about 1.1 M, from about 0.5 M to about 1 M, from about 0.6 M to about 1.4 M, from about 0.6 M to about 1.3 M, from about 0.6 M to about 1.2 M, from about 0.6 M to about 1.1 M, from about 0.6 M to about 1 M, from about 0.7 M to about 1.4 M, from about 0.7 M to about 1.3 M, from about 0.7 M to about 1.2 M, from about 0.7 M to about 1.1 M, from about 0.7 M to about 1 M, from about 0.8 M to about 1.4 M, from about 0.8 M to about 1.3 M, from about 0.8 M to about 1.2 M, from about 0.8 M to about 1.1 M, from about 0.8 M to about 1 M), preferably about 0.8 M.

In some particularly contemplated embodiments, sodium sulfate is present at a concentration of about 0.2 M to about 0.6 M (preferably about 0.4 M) and a pH of about 8.5 to about 9.5 (preferably about 9). In some particularly contemplated embodiments, magnesium sulfate is present at a concentration of about 0.6 M to about 1.0 M (preferably about 0.8 M) and a pH of about 4.5 to about 5.5 or about 8.5 to about 9.75 (preferably about 5 or about 9).

Preferably, the salt solution is an aqueous salt solution. That is to say that the solvent of the salt solution includes water. The solvent of the salt solution may comprise water and a water miscible organic solvent such ethanol, acetone, acetonitrile, or methanol. Typically, the salt solution is free from, or substantially free from, an organic solvent such as a water miscible organic solvent.

### Details of the Method Steps

The method of the invention may include a step of removing extraneous material, such as muscle fibres and ligaments, from the cartilage tissue sample. The step of removing extraneous material may include washing the cartilage tissue with an acid, such as acetic acid, citric acid, lactic acid, or hydrochloric acid. Additionally, or alternatively, the step of removing extraneous material may include washing the cartilage tissue with a base, such as a hydroxide solution. Additionally, or alternatively, the step of removing extraneous material may include washing the cartilage tissue with water, for example the cartilage tissue may be rinsed with, or soaked in, water or subjected to a high-pressure water spray or jet. Additionally, or alternatively, extraneous material may be removed by sonicating the cartilage tissue sample.

Typically, the step of removing extraneous material from the cartilage tissue sample is performed at room temperature, or a temperature below room temperature such as at 4° C. to 10° C.

The step of removing extraneous material from the cartilage tissue sample may be repeated 1, 2, 3 or more times. Each step may use the same method for removing extraneous material or a different method for removing extraneous material from the cartilage tissue sample. For example, a cartilage tissue sample may be washed twice with an acid or washed once with an acid and then washed once with a base.

The method of the invention may also include a step of reducing the cartilage tissue sample into particles. For example, the cartilage tissue may be diced or comminuted into particles. Typically, the cartilage tissue is dehydrated, for example by lyophilization, prior to being reduced into particles. The cartilage tissue may be reduced into particles having a diameter of about 1 mm to about 10 mm, for example, about 1 mm to about 2 mm. Preferably, the step of reducing the cartilage tissue into particles is performed before the step of contacting the cartilage tissue with a salt solution (i.e. step ii of the method).

The method of the invention may also include a step of sterilising the cartilage tissue. Suitable sterilisation methods are those that remove bacterial and viral contaminant from the cartilage tissue without disrupting the native structure of the type II collagen in the cartilage tissue sample. Examples of suitable sterilisation methods include washing the cartilage tissue with an oxidizing agent such as hydrogen peroxide or sodium hypochlorite, or exposing the cartilage tissue to radiation, such as gamma radiation sterilisation, ultraviolet (UV) radiation sterilisation, or electron beam radiation sterilisation.

As previously noted, the cartilage tissue of cartilaginous fish (i.e. a Chondrichthyes) such as shark typically also comprises calcium salts (e.g. calcium phosphate). Chondrichthyes cartilage can contain about 15-25% calcium salts. In some embodiments, for example when using cartilage tissue of cartilaginous fish, the method comprises a demineralisation step, i.e. a step of removing or reducing the amount of calcium salts present in the sample of cartilage tissue. Preferably the demineralisation step occurs prior to step ii).

In some embodiments, prior to step ii) the sample of cartilage tissue is subjected to a demineralisation treatment. In some embodiments, the demineralisation treatment comprises contacting the sample of cartilage tissue with a solution comprising EDTA, hydrochloric acid, phosphoric acid, acetic acid, lactic acid, or any combination of any two or more of these. In some specifically contemplated embodiments, the solution comprises EDTA, acetic acid, and/or lactic acid; preferably acetic acid and/or lactic acid. In some embodiments, the EDTA, hydrochloric acid, phosphoric acid, acetic acid, and/or lactic acid is present at a concentration of at least about 0.1 M, at least about 0.2 M, at least about 0.3 M, at least about 0.4 M, at least about 0.5 M, at least about 0.6 M, at least about 0.7 M, at least about 0.8 M, at least about 0.9 M, at least about 1 M, at least about 1 M, at least about 1.5 M, at least about 2 M, at least about 2.5 M, at least about 3 M, at least about 3.5 M, at least about 4 M, at least about 4.5 M, or at least about 5 M, and useful ranges may be selected between any of these values (for example, from about 0.1 M to about 5 M, from about 0.1 M to about 4 M, from about 0.1 M to about 3 M, from about 0.1 M to about 2 M, from about 0.1 M to about 1 M, from about 0.2 M to about 5 M, from about 0.2 M to about 4 M, from about 0.2 M to about 3 M, from about 0.2 M to about 2 M, from about 0.2 M to about 1 M, from about 0.3 M to about 5 M, from about 0.3 M to about 4 M, from about 0.3 M to about 3 M, from about 0.3 M to about 2 M, from about 0.3 M to about 1 M, from about 0.4 M to about 5 M, from about 0.4 M to about 4 M, from about 0.4 M to about 3 M, from about 0.4 M to about 2 M, from about 0.4 M to about 1 M). Preferably the solution comprises about 0.3 M to about 1.0 M acetic acid and/or lactic acid, more preferably about 0.5 M.

A sterilisation step may be employed before the step of reducing the cartilage tissue sample into particles. Additionally, or alternatively, the cartilage tissue sample may be sterilised after being reduced into particles, for example the tissue may be sterilised after being reduced into particles but before being contacting with the salt solution in step ii) of the method, or sterilised after being reduced into particles and after being contacting with the salt solution in step ii) of the method. The method of the invention may involve one or more sterilisation steps, for example the method may involve one sterilisation step using an oxidising agent and one sterilisation step using radiation.

For the avoidance of doubt, the term “under conditions that allow at least one component of the cartilage tissue to dissolve in the salt solution” in step ii) of the method of the invention is to be understood as conditions that facilitate the dissolution of a proteoglycan, glycosaminoglycan, and/or calcium salt (e.g. calcium phosphate) into the salt solution. Such conditions include, for example, the temperature of the salt solution and cartilage tissue sample, the contact time of the salt solution and cartilage tissue, the volume of the salt solution, and the mass of the cartilage tissue sample. For example, in certain exemplary embodiments, the cartilage tissue sample is exposed to the salt solution for about 6 h, about 12 h, about 18 h, about 24 h, or about 48 h at about 10° C., about 15° C. or about 20° C. For example, the cartilage tissue sample may be exposed to the salt solution for about 24 h at about 20° C. Conditions suitable to dissolve a proteoglycan, glycosaminoglycan, and/or calcium salt (e.g. calcium phosphate) in the cartilage tissue sample may be readily determined by one of ordinary skill in the art.

Typically, step iii) of the method involves subjecting the cartilage tissue and salt solution mixture to centrifuging, filtering and/or gravity separation. This step separates the salt solution and any dissolved components of the cartilage tissue from the insoluble components of the cartilage tissue sample, such as the undenatured type II collagen. The insoluble components of the sample may be further processed (for example, air dried, lyophilised, and/or formulated as a pharmaceutical or dietary composition) to provide a type II collagen composition suitable for administration to an animal, such as a human.

Each of steps ii) and iii) may be performed one or more times. For example, step ii) may be performed using a salt solution disclosed herein and then repeated with a different salt solution disclosed herein. In certain embodiments, step ii) may be performed with a salt solution having a pH of about 4 to about 7, and then repeated with a salt solution having a pH of about 7 to about 9. Salt solutions suitable for step ii) and repeats of step ii) are disclosed herein. Step iii) may be performed once the step ii) repeats have been completed or performed directly following each step ii) performed.

### Type II Collagen Composition

The sample provided following step iii) of the method of the invention is referred to herein as a type II collagen composition. The type II collagen composition obtained by the method of the invention comprises undenatured type II collagen. Undenatured type II collagen has been found to have various health benefits such as improving joint function and reducing joint inflammation. It has also shown promise as a treatment for cartilage-related disorders such as osteoarthrosis and rheumatoid arthritis.

The method of the invention has been found to be particularly effective at providing a type II collagen composition that comprises a high percentage of undenatured type II collagen (for example, at least 5% (w/w) undenatured type II collagen). The type II collagen composition of the invention may therefore be administered at lower doses compared type II collagen compositions that containing a smaller proportion of undenatured type II collagen.

The type II collagen composition of the invention typically comprises about 1% to about 50% (w/w) undenatured type II collagen. For example, the type II collagen may comprise about 1%, 5%, 10%, 20%, 30%, 40%, 50%, 75% or 100% (w/w) undenatured type II collagen. For example, the type II collagen may comprise about 0.1% to about 50%, about 0.2% to about 50%, about 0.5% to about 50%, about 1% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 60%, about 0.2% to about 60%, about 0.5% to about 60%, about 1% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 0.1% to about 70%, about 0.2% to about 70%, about 0.5% to about 70%, about 1% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 0.1% to about 80%, about 0.2% to about 80%, about 0.5% to about 80%, about 1% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 0.1% to about 90%, about 0.2% to about 90%, about 0.5% to about 90%, about 1% to about 90%, about 5% to about 90%, about 10% to about 90%, about 15% to about 90%, about 20% to about 90%, about 25% to about 90%, about 30% to about 90%, about 35% to about 90%, about 40% to about 90%, about 45% to about 90%, about 0.1% to about 99%, about 0.2% to about 99%, about 0.5% to about 99%, about 1% to about 99%, about 5% to about 99%, about 10% to about 99%, bout 15% to about 99%, about 20% to about 99%, about 25% to about 99%, about 30% to about 99%, about 35% to about 99%, about 40% to about 99%, about 45% to about 99%, and about 50% to about 100% (w/w) undenatured type II collagen. Preferably, the type II collagen composition comprises at least 4%, at least 5%, at least 6%, at least 7%, at least 8%, at least 9%, at least 10%, at least 15%, or at least 20% (w/w) undenatured type II collagen. More preferably, at least 5% (w/w) undenatured type II collagen.

In some embodiments, the type II collagen may comprise from about 50% to about 100% (w/w) undenatured type II collagen, such as from about 50% to about 95%, from about 50% to about 90%, from about 55% to about 100%, from about 55% to about 95%, from about 55% to about 90%, from about 60% to about 100%, from about 60% to about 95%, from about 60% to about 90%, from about 65% to about 100%, from about 65% to about 95%, from about 65% to about 90%, from about 70% to about 100%, from about 70% to about 95%, from about 70% to about 90%, from about 75% to about 100%, from about 75% to about 95%, from about 75% to about 90%, from about 80% to about 100%, from about 80% to about 95%, or from about 80% to about 90% (w/w) undenatured type II collagen.

The undenatured type II collagen content of the composition of the invention may be determined using methods known in the art, for example, by the ELISA based method described by Harris et al., (J. Diet Suppl. 2021, 1:1-16) or by a trypsin digestion susceptibility assays such as those described by Veres et al., and Bank et al., (Matrix Biol, 2014, 33:54-59 and Matrix Biol, 1997, 16(5): 233-243). Or, for example, the undenatured type II collagen content may be determined by measuring the average molecular weight of the collagen in the composition as described herein.

For the avoidance of doubt, the term “w/w” as used herein refers to weight for weight of a particular substance within a mixture. For example, a type II collagen composition containing 10% (w/w) undenatured type II collagen is to be understood as containing 10 grams of undenatured type II collagen for every 100 grams of the type II collagen composition. Unless stated otherwise, any reference herein to the mass of type II cartilage, undenatured type II collagen, or cartilage tissue sample is to be understood as referring to the dry mass of the type II cartilage or cartilage tissue sample.

As described previously, undenatured type II collagen is typically insoluble in aqueous solutions whereas denatured and/or hydrolysed type II collagen is soluble in aqueous solutions. The solubility of a type II collagen composition is determined as described in Example 6. In some embodiments, the type II collagen composition of the invention has a solubility of less than about 80%, such as less than less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 4%, less than about 3%, less than about 2%, less than about 1%, or about 0%, and useful ranges may be selected between any of these values (for example, from about 0% to about 70%, from about 0% to about 50%, from about 0% to about 30%, from about 0% to about 10%, from about 0% to about 5%, from about 0% to about 4%, from about 0% to about 3%, from about 1% to about 70%, from about 1% to about 50%, from about 1% to about 30%, from about 1% to about 10%, from about 1% to about 5%, from about 1% to about 4%, from about 1% to about 3%, from about 2% to about 70%, from about 2% to about 50%, from about 2% to about 30%, from about 2% to about 10%, from about 2% to about 5%, from about 2% to about 4%, from about 2% to about 3%, from about 3% to about 70%, from about 3% to about 50%, from about 3% to about 30%, from about 3% to about 10%, or from about 3% to about 5%).

In some embodiments, the method does not comprise treatment with a protease. In some embodiments, the purified type II collagen has not been treated with a protease.

In certain applications it may be desirable to produce soluble type II collagen that has maintained its triple helical structure. The type II collagen composition may be treated with a proteolytic enzyme such as pepsin to cleave the N- and C-terminal telopeptides, liberating soluble, triple helical type II collagen, termed ‘atelocollagen’.

The applicant has discovered that to effectively produce atelocollagen, GAGs must first be removed. Thus, the methods of the invention can enhance the extraction of atelocollagen from cartilage tissue. Further, when GAGs are removed using chaotropic agents such as guanidinium hydrochloride or urea, the chaotropic agents may need to be removed before treatment with the proteolytic enzyme, or they may interfere with proteolysis. In contrast, without wishing to be bound by theory, it is believed that the methods of the invention may allow subsequent proteolytic treatment without the need for (or with a reduced need for) removing the extraction reagent(s).

In some embodiments, the method for purifying type II collagen from cartilage tissue further comprises: iv) treating the sample with a proteolytic enzyme (preferably pepsin) to produce atelocollagen. In some embodiments, the method produces at least about 30 μg of atelocollagen per mg cartilage, such as at least about 35 μg, at least about 40 μg, at least about 45 μg, at least about 50 μg, at least about 55 μg, at least about 60 μg, at least about 65 μg, at least about 70 μg, at least about 75 μg, at least about 80 μg, at least about 85 μg, at least about 90 μg, at least about 95 μg, or at least about 100 μg of atelocollagen per mg cartilage, and useful ranges may be selected between any of these values (for example, from about 30 to about 100, from about 30 to about 90, from about 30 to about 80, from about 35 to about 100, from about 35 to about 90, from about 35 to about 80, from about 40 to about 100, from about 40 to about 90, from about 40 to about 80, from about 45 to about 100, from about 45 to about 90, from about 45 to about 80, from about 50 to about 100, from about 50 to about 90, from about 50 to about 80, from about 55 to about 100, from about 55 to about 90, from about 55 to about 80, from about 60 to about 100, from about 60 to about 90, from about 60 to about 80, from about 65 to about 100, from about 65 to about 90, from about 65 to about 80, from about 70 to about 100, from about 70 to about 90, from about 70 to about 80, from about 75 to about 100, from about 75 to about 90, or from about 75 to about 80).

Also provided is atelocollagen obtained by this method.

The type II collagen composition obtained from the method of the invention may comprise proteoglycan, glycosaminoglycan, and/or calcium salt (e.g. calcium phosphate). Preferably, the proteoglycan, glycosaminoglycan, and/or calcium salt (e.g. calcium phosphate) content of the type II collagen composition is less than the proteoglycan, glycosaminoglycan, and/or calcium salt (e.g. calcium phosphate) content of the cartilage tissue sample before being subjected to the method of the invention.

In exemplary embodiments, the type II collagen composition of the invention contains less than 50% (w/w) proteoglycans and/or glycosaminoglycans. For example, less than 45%, less than 40%, less than 30%, less than 20%, less than 10%, less than 5%, less than 2.5%, or less than 1% (w/w) proteoglycans and/or glycosaminoglycans. In certain embodiments, the type II collagen composition of the invention contains less than about 45% (w/w) proteoglycans and/or glycosaminoglycans.

In certain embodiments, the type II collagen composition of the invention contains less than 50% (w/w) calcium salt (e.g. calcium phosphate). For example, less than 45%, less than 40%, less than 30%, less than 20%, less than 10%, less than 5%, less than 2.5%, or less than 1% (w/w) calcium salt (e.g. calcium phosphate).

Typically, the type II collagen composition of the invention comprises less than 20%, less than 15%, less than 10%, less than 5%, less than 2.5%, or less than 1% (w/w) water. Preferably, less than 10%, less than 5%, less than 2.5%, or less than 1% (w/w) water. For example, the type II collagen composition of the invention may be substantially free from water.

While it is possible for the type II collagen composition of the invention to be administered alone, it is preferable for it to be formulated as a composition suitable for human and/or animal consumption.

The type II collagen composition of the invention may be incorporated into a variety of formulations for therapeutic administration. More particularly, the type II collagen composition of the invention may be formulated as a pharmaceutical composition by combination of the type II collagen composition with appropriate pharmaceutically acceptable carriers or diluents.

Thus, in certain embodiments, the type II collagen composition of the invention comprises one or more carriers (including excipients and diluents), for example one or more pharmaceutically acceptable carriers (including pharmaceutically acceptable excipients and diluents).

Various pharmaceutically acceptable carriers and formulations are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

For oral administration, the type II collagen composition may be administered alone or in combination with one or more carriers or diluents to make capsules, tablets, edible film, lozenge, and powders. The type II collagen composition may also be presented as a bolus, electuary or paste. The compositions may optionally be present in a form that provides slow or controlled release of the type II collagen once administered to a subject.

Pharmaceutical compositions suitable for therapeutic administration include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration, although the most suitable route may depend upon the characteristics of the subject being administered the composition, for example the species, age, weight, sex, medical conditions, the severity of the medical conditions, and other relevant medical and physical factors.

Compositions for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The composition may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. Compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Compositions for rectal administration may be presented as a suppository with carriers such as cocoa butter, synthetic glyceride esters or polyethylene glycol. Such carriers are typically solid at ordinary temperatures, but liquefy and/or dissolve in the rectal cavity to release the drug.

Compositions for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia. Exemplary compositions for topical administration include a topical carrier such as Plastibase (mineral oil gelled with polyethylene).

The type II collagen composition of the invention may also be formulated as a food additive, food ingredient, health functional food, dietary supplement, medical food, nutraceutical, or food supplement. For example, it may be presented in the form of a beverage, food bar, syrup, spread, sauce, paste, jelly, pudding, soup base, capsule, tablet, edible film, lozenge, or powder. In exemplary embodiments, the type II collagen composition comprises one or more excipient selected from the group consisting of microcrystalline cellulose, stearic acid and silicon dioxide.

In certain exemplary embodiments, the type II collagen composition may in the form of a tablet with a core and a coating, wherein the core comprises the type II collagen, microcrystalline cellulose, stearic acid and silicon dioxide, and the coating comprises hypromellose.

It should be understood that in addition to the ingredients mentioned above, the composition of the invention may include other agents conventional in the art having regard to the type of composition in question. In certain embodiments, the composition may comprise one or more further therapeutic agents or dietary supplement. Examples of further therapeutic agents and dietary supplements that may be present in a composition of the invention include, but not limited to, antihistamines, anti-inflammatoires, disease-modifying antirheumatic drugs (DMARDs), corticosteroids, non-steroidal anti-inflammatory drugs (NSAIDs), glucosamine, chondroitin, and S-adenosyl-methionine.

### Treatments

The present invention provides a type II collagen composition for use as a medicament and/or dietary supplement. In certain embodiments, the type II collagen composition of the invention may be administered to a subject suffering from cartilage-related disorders, such osteoarthrosis and rheumatoid arthritis. Alternativity, or additionally, the type II collagen composition may be administered to a subject known or suspected of being at risk of developing a cartilage-related disorder.

A “cartilage-related disorder” herein refers to disorders relating to the function of cartilage tissue in the human or animal body. Examples of cartilage-related disorders include joint stiffness, joint discomfort, joint inflammation, reduced joint mobility, exercise-induced joint pain, and arthritis (for example, osteoarthritis and rheumatoid arthritis. Further examples include bursitis, gout, tendonitis, joint sprains and joint strains.

In certain embodiments, the type II collagen composition may be consumed by a subject as a dietary supplement to promote joint health, for example by reducing or preventing joint stiffness, joint discomfort and/or improve joint mobility.

The present invention further provides a method for treating or preventing cartilage-related disorders, wherein said method comprises a step of administering a dose of the type II collagen composition of the invention to a subject in need thereof. The present invention also provides a type II collagen composition for use in the manufacture of a medicament, for example a medicament for use in treatment or prevention of cartilage-related disorders.

For the avoidance of doubt, the term “subject” as used herein refers to a living vertebrate. In certain embodiments, the subject is a mammal, for example the subject may be a human. In exemplary embodiments, the subject is a human.

A therapeutic dose of the type II collagen composition prepared according to the present invention is a dose sufficient to treat or prevent a cartilage-related disorders. By way of example, the composition may be administered in dosages of from 0.1 mg or less up to 5000 mg or more per day. An exemplary human dose may be from 1 mg to 200 mg per day. The dosage may be determined based on the total type II collagen content of the composition or the total undenatured type II collagen content of the composition. Preferably, the dosage is determined based on the total undenatured type II collagen content of the composition. It should be appreciated that type II collagen may be administered as a single daily dose or administer as a number of discrete divided doses as may be appropriate. An ordinarily skilled physician can readily determine and administer a therapeutic dose, or an appropriate fraction thereof, required to have the desired therapeutic effect.

### Numbered Paragraphs

The present disclosure will now be further described by way of the following numbered paragraphs.

1. A method for purifying type II collagen from cartilage tissue, said method comprising:
i) providing a sample of cartilage tissue;
ii) contacting the sample with a salt solution having a pH of about 3 to about 9 under conditions that allow at least one component of the cartilage tissue to dissolve in the salt solution, wherein the at least one component of the cartilage tissue is selected from the group consisting of proteoglycan, glycosaminoglycan, and calcium salt; and
iii) removing the salt solution and the at least one component of the cartilage tissue from the sample.

2. The method of paragraph 1, wherein the salt solution has a pH of about 3.5 to about 4.5, for example a pH of about 4.

3. The method of paragraph 1 or paragraph 2, wherein the salt solution is a sulfate salt solution or chloride salt solution.

4. The method of any one of paragraph 1 to 3, wherein the salt solution has a sulfate or chloride concentration of about 0.4 M to about 2 M.

5. The method of any one of paragraph 1 to 4, wherein the salt solution is an aqueous salt solution.

6. The method of any one of paragraph 1 to 5, wherein the sample of cartilage tissue has been obtained from a fish, bird, mammal, mollusc, or echinoderm.

7. The method of any one of paragraph 1 to 6, wherein the sample of cartilage tissue is from a fish.

8. The method of paragraph 7, wherein the fish is a bony fish, for example the fish is a cod, haddock, pollock, salmon (for example, Salmo salar), European hake (also known as a Merluccius merluccius) or blue grenadier (also known as a Macruronus novaezelandiae or hoke).

9. The method of paragraph 7, wherein the fish is a cartilaginous fish, for example a shark, ray, skate, or sawfish.

10. The method of paragraph 7 or 8, wherein the component dissolved by the salt solution is a glycosaminoglycan.

11. The method of paragraph 9, wherein the component dissolved by the salt solution is calcium salt, for example calcium phosphate.

12. The method of any one of paragraph 1 to 11, wherein step iii) removes about 10% to about 100% of the proteoglycan, glycosaminoglycan, and/or calcium salt from the sample.

13. The method of any one of paragraph 1 to 12, wherein the percentage (w/w) of type II collagen in the sample is about 40% to about 100% greater than the percentage (w/w) of type II collagen in the sample before step iii), and preferably the percentage (w/w) of undenatured type II collagen in the sample is about 40% to about 100% greater than the percentage (w/w) of undenatured type II collagen in the sample before step iii).

14. A type II collagen composition obtained by the method of any one of paragraph 1 to 13.

15. The composition of paragraph 14 comprising a pharmaceutically acceptable excipient.

16. The composition of paragraph 13 or paragraph 14 for use as a medicament.

17. The composition of paragraph 13 or paragraph 14 for use in the treatment or prevention of a cartilage-related disorder.

18. Use of the composition of paragraph 13 or paragraph 14 as a dietary supplement.

19. A method for the treatment or prevention of a cartilage-related disorder, said method comprising the step of administering a dose of the composition of paragraph 13 or paragraph 14 to a subject in need thereof.

20. Use of the composition of paragraph 13 or paragraph 14 for the manufacture of a medicament.

21. The use of paragraph 20, wherein the medicament is to be used for the treatment of a cartilage-related disorder.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

### Example 1: Purification Method Development

### Cartilage Tissue Processing Method:

Chicken carcasses and hoki heads were bought from a butcher and fishing company, respectively. Chicken sternum cartilage and hoki nasal cartilage was removed by hand and washed in 0.1 M acetic acid overnight with mixing to remove extraneous ligaments/skin at 10° C. Cartilage was recovered using a sieve and rinsed with water.

The cartilage samples were lyophilised and ground into 1 mm particles using a blender followed by a mill.

Fifty mg of lyophilised and ground cartilage tissue sample was mixed with 1 mL of an extraction reagent (4 M guanidinium hydrochloride solution; 1%, 0.25% or 0.1% trypsin solution; 2% tributyl phosphate solution; 0.1-0.4 M sodium sulfate solution pH 3.9-10; 0.1-0.2 M sodium acetate solution pH 3.9-10; 2% triton-X solution; 2% CHAPs solution; or 0.1 M acetic acid solution). The mixtures were mixed end-over-end at 20° C. or 40° C. for 24 h. The samples were then centrifuged (17,000×g, 5 min) and the supernatant was transferred into another tube. The pelleted material from this method step is referred to hereinafter as Pellet A.

GAGs were precipitated from the supernatant by adding 500 μL acetone. The precipitate was pelleted using centrifugation and the supernatant was removed. The pelleted material from this method step is referred to hereinafter as Pellet B. Pellet B was resuspended in phosphate buffered saline buffer (pH 7.4) before analysis.

### 1a) Glycosaminoglycan Extraction:

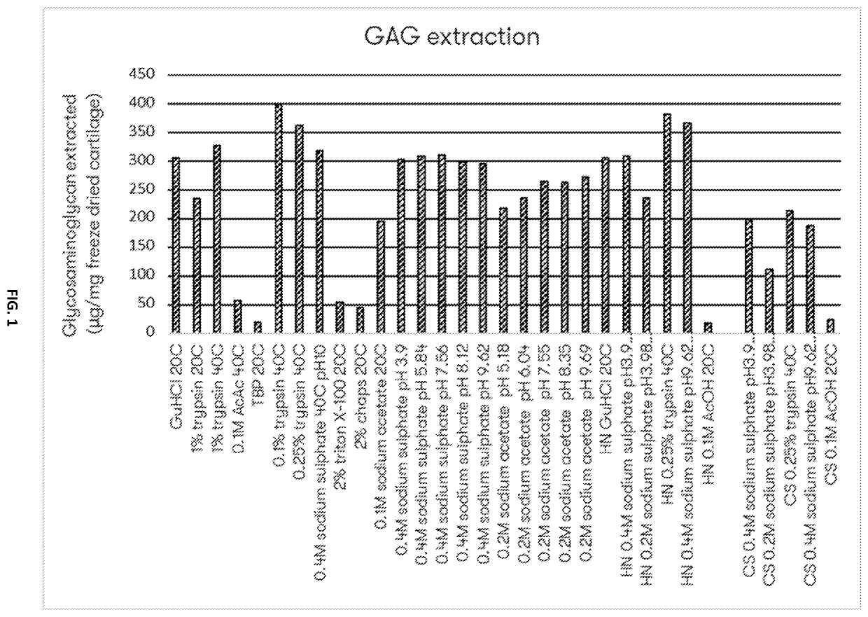

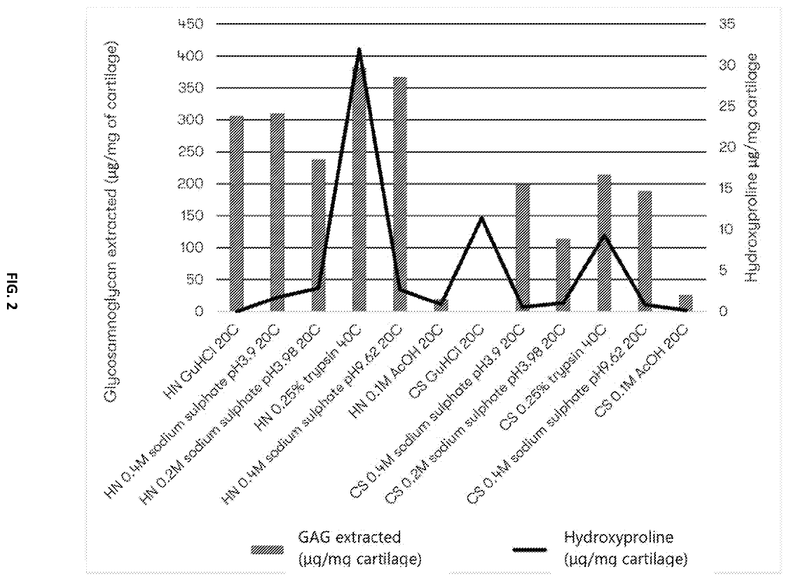

The amount of GAG in Pellet B was measured using the dimethylene blue assay (DMMB) using a method based on that described by Coulson-Thomas and Gesteira (Bio-protocol, 4(18): e1236, which is hereby incorporated herein by reference). The resuspended Pellet B samples were compared to a standard curve of chondroitin 4 sulfate. The amounts of GAG extracted from the cartilage tissue samples are expressed in FIGS. 1 and 2 as μg of GAG per mL of extraction reagent normalised by the mass of lyophilised and ground cartilage tissue used in the extraction.

### Results:

The extraction reagents and conditions (i.e. 20° C. or 40° C.) used were effective at extracting GAGs from hoki nose cartilage or chicken sternum cartilage (see FIG. 1). Extractions using a sodium sulphate solution or sodium acetate solution were found to extract similar amounts of GAGs to the extractions that used a trypsin or guanidinium hydrochloride solution.

### 1b) Collagen Hydrolysis in Cartilage Sample:

Hydroxyproline is an amino acid that is found almost exclusively in collagen. Hydroxyproline extracted from a cartilage tissue sample following treatment with an extraction reagent is a sign of collagen hydrolysis, which may indicate a reduction in undenatured collagen in the sample. A high level of GAG extraction and low level of hydroxyproline extraction suggest effective GAG removal by an extraction reagent without disruption of the undenatured collagen content of a sample.

Hydroxyproline levels were measured using the HPLC method described by Henderson et al. (Rapid, Accurate, Sensitive and Reproducible HPLC Analysis of Amino Acids. Agilent Technologies, Application Note, Publication No: 5980-1193). Samples were hydrolysed in 6 M HCl and derivatized with fluorenylmethyloxycarbonyl chloride (FMOC-CI) before being run on a Kinetex® 2.6 μm EVO C18 100 Å column. The florescence of the FMOC group was measured with the ex./em. 266/305 nm. The area of the peak corresponding to FMOC-hydroxyproline was measured against FMOC-hydroxyproline of known amount.

### Results:

Sodium sulfate extraction reagents were found to extract high levels of GAGs and low levels of hydroxyproline (see FIG. 2), indicating that the extraction conditions did not disrupt the collagen contained in the samples. This contrasts with the extraction conditions using a trypsin solution, which resulted in high levels of GAG extraction and hydroxyproline, indicating that trypsin hydrolysed the collagen in the sample into hydroxyproline.

### Example 2: Evaluation of the Sodium Sulphate Extraction Reagent

Hoki nasal, dog fish spine, and chicken sternum cartilage samples were processed using the cartilage tissue processing method described in Example 1. Fifty mg of lyophilised and ground cartilage tissue sample was mixed with 1 mL of the extraction reagent (0.4 M sodium sulphate solution, pH 4) at 20° C. for 20 h with end-over-end mixing.

Following treatment with the extraction reagent, the samples were processed according to the cartilage tissue processing method described in Example 1 to provide a Pellet A and Pellet B for analyses.

2a) Glycosaminoglycan Extraction from Cartilage Sample:

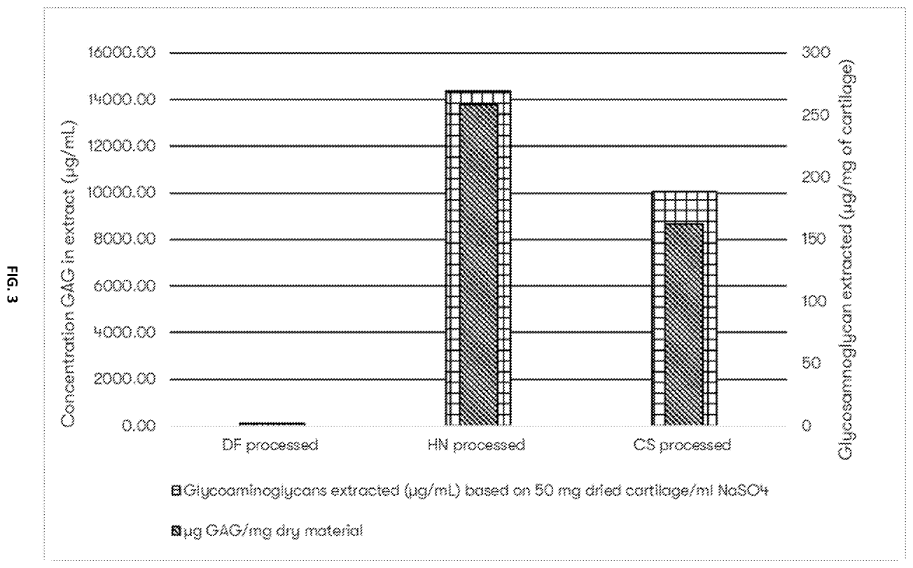

The amount of GAG in Pellet B obtained from the hoki nasal, dog fish spine, and chicken sternum cartilage tissue samples were analysed using the method described in Example 1a. The amount of GAG extracted from each cartilage tissue sample is expressed in FIG. 3 as μg of GAG per mL of extraction reagent (left-hand y-axis), or μg of GAG per mL of extraction reagent normalised by the mass of lyophilised and ground cartilage tissue used in the extraction (right-hand y-axis).

### Results:

The extraction reagent was found to extract approx. 30 mg and approx. 50 mg GAGs per gram of lyophilised and ground chicken sternum and hoki nasal cartilage tissue, respectively. No notable amount of GAG was extracted from lyophilised and ground dogfish cartilage tissue (see FIG. 3).

### 2b) Collagen Content in Cartilage Samples:

The amount of undenatured collagen in the pelleted cartilage tissue samples following the extraction method (i.e. the Pellet A samples) was measured using a trypsin digestion susceptibility assay. Undenatured collagen is more resistant to hydrolysis by trypsin than denatured collagen. A sample with a high level of undenatured collagen would be expected to have a low level of free hydroxyproline in the sample following trypsin treatment.

The assay method was based on the methods described by Veres et al., and Bank et al, (Matrix Biol 33:54-59 and Matrix Biol 16(5): 233-243 which are hereby incorporated herein by reference). In brief, the Pellet A samples produced by the extraction method (i.e. treatment of cartilage tissue with 0.4 M sodium sulphate solution (pH 3.9) at 20° C. for 18 h with end-over-end mixing) were lyophilised to ensure an accurate weight of each sample could be measured. As a control, a lyophilised and ground cartilage tissue sample that had not undergone the extraction method was used. UCII® (Lonza) was used as a further control. The UCII® sample used was reported as containing 40 mg cartilage with 1.2 mg of undenatured type II collagen. The Pellet A samples, and the UC-∥® sample, were mixed with 14,7000 Units of porcine trypsin (type IX-S) per mg in 100 mM phosphate buffer pH 7.4, and digested at 20° C. for 24 h. The reactions were centrifuged, and the supernatant was immediately removed and frozen until analysis. The pellet and supernatant were hydrolysed in 6 N HCl, 110° C. for 24 h followed by measurement of the hydroxyproline content. The amount of hydroxyproline in the pellet following the trypsin treatment is an indirect measure of undenatured collagen content in the pellet, and therefore in the cartilage tissue sample following the extraction method. The sum of the amount of hydroxyproline in the pellet and supernatant is an indirect measure of the total collagen content of a cartilage tissue sample following the extraction method.

### Results:

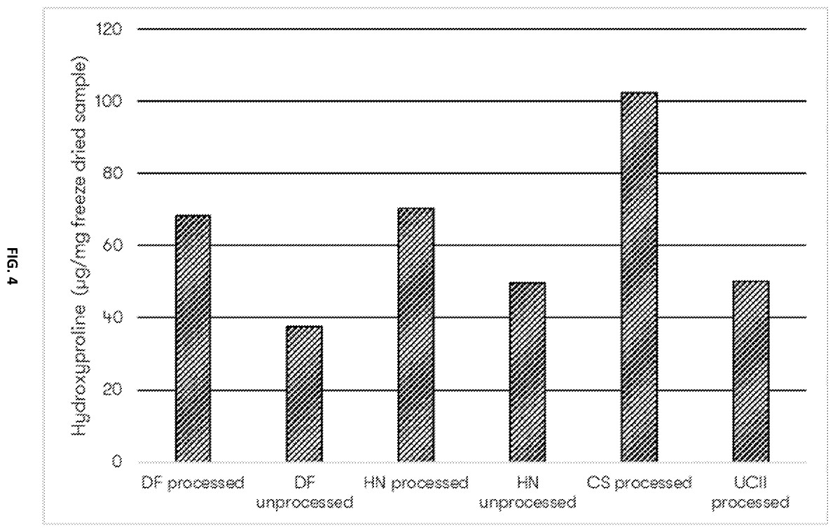

The cartilage tissue samples subjected to the extraction method (i.e. “processed” samples) were found to contain more collagen per mg of dry sample compared to the control samples that had not undergone the extraction method (i.e. “unprocessed” samples), suggesting that the extraction method removed non-collagen components (e.g. GAGs and calcium phosphate) from the cartilage (see FIG. 4). In particular, the processed dogfish samples (DF processed) had 1.7 times higher collagen content compared to the unprocessed dogfish cartilage tissue samples (DF unprocessed); the processed hoki nasal samples (HN processed) had 1.45 times higher collagen content compared to the unprocessed hoki nasal samples (HN unprocessed); and the processed treated chicken sternum samples (CS processed) had 2.2 times higher collagen content compared to the UCII® sample (see FIG. 4). Applying a conversion factor based on the hydroxyproline content of pure type II collagen (9.5 for marine and 10.22 for chicken type II collagen) to convert hydroxyproline (μg/mg dry sample) to percentage collagen, the percentage collagen in each cartilage sample was estimated to be from about 35% to about 100%, with the lowest being in unprocessed dogfish cartilage tissue samples and UCII®, and highest being in processed chicken sternum cartilage tissue samples (Cumming et al., 2019, Marine Drugs 17(4): 223, Naffa et al., 2019. Journal American Leather Chemists Association 114:29-37, and Don et al., 2013, Modern Food Science and Technology 29:2538-2541+2419).

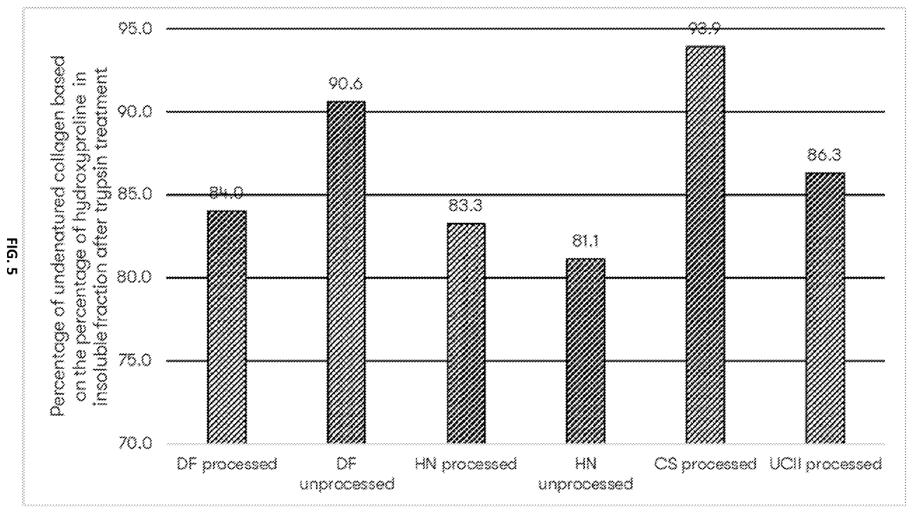

FIG. 5 shows that all samples subjected to the extraction method (i.e. “processed” samples) maintained a high percentage of undenatured collagen relative to total collagen. content than the control samples that had not undergone the extraction method (i.e. “unprocessed” samples).

### Example 3: The Effect of Salt and pH on Undenatured Collagen Content and GAG Extraction from Cartilage Tissue

### 3a) Cartilage Tissue Processing Method:

Hoki (blue grenadier, also known as Macruronus novaezelandiae) nasal cartilage and chicken sternum were removed by hand from frozen carcasses. The extracted cartilage tissue samples were then washed with 0.5 M acetic acid overnight at 10° C. to remove ligaments and extraneous tissue. The cartilage tissue samples were then frozen and lyophilised. The lyophilised material was then ground into 1 mm to 2 mm particles.

One millilitre of extraction reagent was added to 50 mg of lyophilised and ground cartilage tissue sample. The extraction reagents used were solutions of sodium, ammonium or magnesium chloride salts; or solutions of sodium, ammonium or magnesium sulfate salts. Each salt solution was tested at a 0.5 M or 1 M salt concentration with a pH of 4, 7 or 9. The mixture was mixed end-over-end at 20° C. for 24 h. The mixtures were then centrifuged (17,000×g, 5 min). The supernatant was removed and analysed for glycosaminoglycan content as described in 3b). The pelleted material was analysed for undenatured collagen content as described in 3c).

3b) Glycosaminoglycan Extraction from Cartilage Sample:

Glycosaminoglycans in the supernatant removed from the processed cartilage tissue sample were precipitated by adding 500 μL acetone to each supernatant sample. The precipitates were pelleted using centrifugation. The pellets were resuspended in phosphate buffered saline buffer, pH 7.4, the glycosaminoglycans content measured using the dimethylmethylene (DMMB) blue assay according to the method described by Coulson-Thomas et al. (Bio-protocol, 2014, Vol 4, Iss 18). The readings were compared against a standard curve of chondroitin 4-sulfate.

### 3c) Collagen Content in Cartilage Sample:

The amount of undenatured collagen in the pelleted cartilage tissue samples treated with a 1 M magnesium sulfate solution having a pH of about 4, 7 or 9, or with a 0.5 M sodium sulfate solution was indirectly measured using the trypsin digestion susceptibility assay. The assay method was the same as that described in Example 2b).

### Results:

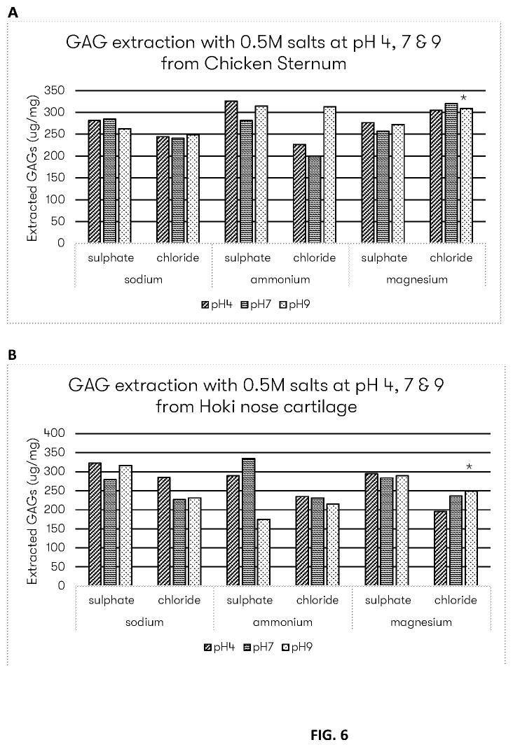

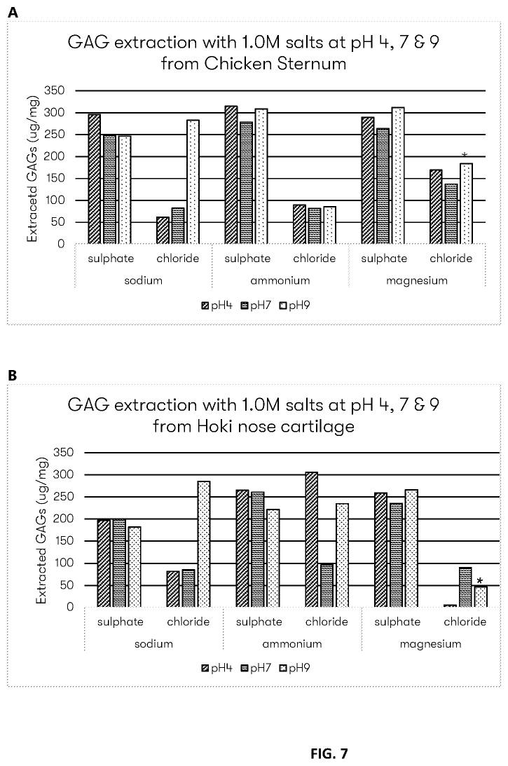

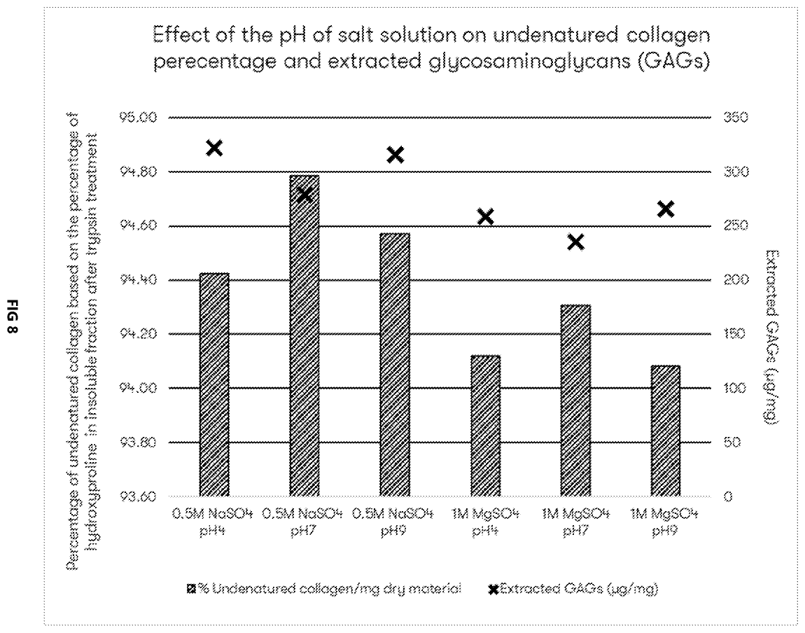

The sulfate salts (sodium sulfate, ammonium sulfate and magnesium sulfate) and chloride salts (sodium chloride, ammonium chloride and magnesium chloride) tested were found to extract GAG from hoki nose cartilage and chicken sternum cartilage. The sulphate salts were generally found to be more effective than the chloride salts at extracting GAGs (see FIGS. 6 and 7). Hoki nose cartilage tissue samples treated with an extraction reagent having a pH of about 4 or 7 were found to contain higher levels of undenatured collagen than samples treated with an extraction reagent having a pH of about 9. Salt solutions with a pH of about 4 were found to extract GAGs from the cartilage tissue samples whilst maintaining good levels of undenatured collagen in the cartilage tissue samples (see FIG. 8).

### Example 4: Effect of Salt Concentration and pH on GAG Extraction

Hoki nasal cartilage was processed using the cartilage tissue processing method described in Example 1, using Na₂SO₄ or MgSO₄ as the extraction reagent at a variety of concentrations and pHs. The amount of GAG extracted was measured as described in Example 1.

### Results

GAGs were effectively extracted from the cartilage tissue over a broad range of salt concentrations and pHs as shown in Tables 1 and 2.

TABLE 1
Effect of salt concentration on GAG extraction.

| | |
|---|---|
| Salt concentration | GAGs extracted (μg/mg cartilage) |

| (M) | Na₂SO₄, pH 4 | MgSO₄, pH 4 |
|---|---|---|
| 0 | 6.75 | 7.27 |
| 0.2 | 204.57 | 141.39 |
| 0.4 | 242.99 | 193.47 |
| 0.6 | 209.30 | 222.98 |
| 0.8 | 201.40 | 227.01 |
| 1.0 | 150.76 | 224.92 |
| 1.2 | 112.65 | 200.12 |
| 1.4 | 91.13 | 188.27 |

TABLE 2
Effect of pH on GAG extraction.

GAGs extracted (μg/mg cartilage)

| pH | 0.4M Na₂SO₄ | 0.8M MgSO₄ |
|---|---|---|
| 3 | 215.42 | 193.70 |
| 4 | 215.66 | 217.45 |
| 5 | 199.45 | 221.77 |
| 6 | 200.34 | 201.91 |
| 7 | 205.94 | 198.20 |
| 8 | 204.41 | 204.94 |
| 9 | 232.49 | 221.07 |
| 9.75* | | 220.47 |
| 10 | 210.72 | |

*0.8M MgSO₄ was measured at pH 9.75 instead of pH 10 due to reduced solubility of MgSO₄ at high pH.

### Example 5: Cartilage Sources

### 5a) GAG Content of Cartilage Sources

The total amount of GAG per mg freeze dried cartilage was determined for hoki nose, dogfish (mineralised cartilage), chicken sternum, lamb trachea, veal trachea, and bovine joint cartilage. Briefly, a known amount of tissue was treated with a proteolytic enzyme (the endo-protease, Alcalase®) for 24 hours to fully solubilise all proteinaceous material. GAGs were precipitated from the solution by adding 5 volumes acetone per 1 volume sample. The precipitate was recovered by centrifugation and resuspended in phosphate buffer, and the amount of GAGs measured using the dimethylmethylene blue (DMMB) assay.

The efficacy of GAG extraction using various sources of cartilage was tested. Cartilage from chicken sternum, lamb trachea, veal trachea, and bovine joint was prepared as described in Example 1, using 0.4 M Na₂SO₄ at pH 4 or 9, or 0.8 M MgSO₄ at pH 5 or 9, as the extraction reagent. The amount of GAG extracted per mg cartilage was quantified as described in Example 1. The percentage of undenatured collagen was determined based on the percentage of hydroxyproline in the insoluble fraction after trypsin treatment, as described in Example 2b.

### Results

The GAG content of various cartilage sources is shown in Table 3.

TABLE 3
GAG content of various cartilage sources.

| | GAG content (μg/mg |
|---|---|
| Cartilage source | freeze dried cartilage) |
| Hoki nose | 430.1 |
| Dogfish (mineralised cartilage) | 87.8 |
| Lamb trachea | 187.47 |
| Veal trachea | 325.90 |
| Bovine knee joint | 183.72 |
| Chicken sternum | 348.71 |

The efficacy of Na₂SO₄ and MgSO₄ in extracting GAGs from various cartilage sources is shown in Table 4.

TABLE 4
GAG extraction from other cartilage sources.

GAGs extracted (μg/mg cartilage)

| | 0.4M | 0.4M | 0.8M | 0.8M |
|---|---|---|---|---|
| Cartilage | Na₂SO₄ | Na₂SO₄ | MgSO₄ | MgSO₄ |
| source | pH 4 | pH 9 | pH 5 | pH 9 |
| Chicken sternum | 225.62 | 231.89 | 227.80 | 224.70 |
| Lamb trachea | 75.56 | 73.70 | 76.51 | 75.61 |
| Veal trachea | 41.95 | 40.40 | 31.81 | 27.69 |
| Bovine joint | 121.39 | 115.33 | 113.19 | 112.14 |

The undenatured collagen content (as a percentage of total collagen) of samples of chicken sternum and bovine joint cartilage extracted with 0.4 M Na₂SO₄ pH4, and 0.8 M MgSO₄ pH9 is shown in Table 5.

TABLE 5
Percentage undenatured collagen.

| | Percentage of collagen that is undenatured (w/w) | | |
|---|---|---|---|
| Cartilage | 0.4M | 0.8M | |
| source | Na₂SO₄ pH 4 | MgSO₄ pH 5 | Water |
| Chicken sternum | 88.05 | 89.15 | 83.14 |
| Bovine joint | 98.20 | 98.50 | 98.46 |

### 5b) Dogfish Cartilage

The cartilage tissue of cartilaginous fish (i.e. Chondrichthyes, such as sharks) comprises calcium salts (e.g. calcium phosphate). Chondrichthyes cartilage can contain about 15-25% calcium salts. Demineralisation treatments were tested for their effect on GAG removal and denaturation of dogfish cartilage.

Dogfish cartilage was pre-treated using a variety of demineralisation treatments, and then processed using the cartilage tissue processing method described in Example 1, using 1M MgSO₄ pH4 as the extraction reagent. The amount of GAG extracted was measured as described in Example 1, and the percentage of undenatured collagen was measured as described in Example 2.

### Results

The effect of various demineralisation pre-treatments on GAG extraction from dogfish cartilage using 1M MgSO₄ pH4 is shown in Table 6. Undenatured collagen is shown as a percentage of total collagen.

TABLE 6
Effect of demineralisation treatments on
GAG extraction from dogfish cartilage.

| | GAGs extracted | Percentage of |
|---|---|---|
| Demineralisation | (μg/mg | collagen that is |
| treatment | cartilage) | undenatured (w/w) |
| Water | 3.13 | 92.0 |
| 0.5M EDTA | 4.76 | 90.3 |
| 0.5M HCl | 0.36 | 84.7 |
| 0.5M phosphoric acid | 1.91 | 84.7 |
| 0.5M acetic acid | 20.25 | 88.5 |
| 0.5M lactic acid | 15.97 | 85.3 |

### Example 6: Comparative Extraction Methods

Samples of freeze dried hoki cartilage were processed using the cartilage tissue processing method described in Example 1, using 1M MgSO₄ at pH4 or pH9 as the extraction reagent.

Comparative Samples were Processed by the Following Method:

Milled freeze dried hoki cartilage was treated with 50 mM Tris pH 8, 4 M guanidinium hydrochloride, 0.1% PMSF, 2 mM EDTA for five days to remove proteoglycans. The insoluble material was pelleted by centrifugation, washed twice with 0.1 M acetic acid, and treated with 0.1% pepsin in 0.2 M acetic acid for 24 hours. The soluble fraction was dialysed against 0.1 M acetic acid, and the collagen precipitated using sodium chloride.

The percentage of soluble protein in the collagen samples was determined by suspending in 0.5 M acetic acid and incubating at 20° C. for 1 hour with mixing. These were then centrifuged at 3260×g for 15 minutes and the protein concentration of the supernatant measured using the biuret assay. Samples of collagen were also visualised by scanning electron microscopy.

### Results

The percentages of soluble protein in untreated hoki cartilage, collagen prepared using 1M MgSO₄ as described in Example 1, and the comparative pepsin-treated collagen are shown in Table 7. The native (untreated) cartilage and the collagen prepared using MgSO₄ were highly insoluble, whereas the collagen prepared using pepsin digestion was highly soluble.

TABLE 7
Percentage of soluble protein.

| Treatment | Percentage soluble protein |
|---|---|
| Untreated hoki cartilage | 1.49 |
| 1M MgSO₄ pH 4 | 3.35 |
| 1M MgSO₄ pH 9 | 0.00 |
| Pepsin | 86.32 |

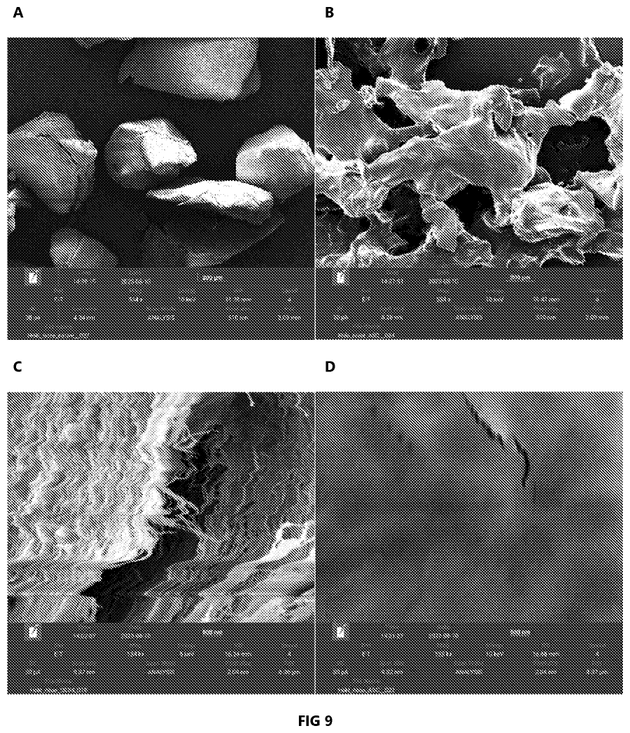

Scanning electron micrographs of the MgSO₄ and pepsin-treated collagen are shown in FIG. 9. At low magnification (534×), the MgSO₄-treated collagen appears as discrete particles, whereas the pepsin-treated collagen appears as irregular blobs. At high magnification (133,000×), the MgSO₄-treated collagen appears fibrillar, whereas the pepsin-treated collagen lacks any visible fibrillar structure.

### Example 7: Salt Treatment Increases Yield of Pepsin-Soluble Type II Collagen (Atelocollagen)

To prepare soluble type II collagen that has maintained its triple helical structure, pepsin is used to cleave the N- and C-terminal telopeptides, liberating the triple helical type II collagen. This is termed atelocollagen. For effective collagen extraction, glycosaminoglycans must first be removed.

Various extraction reagents were tested for their ability to remove GAGs and produce triple helical type II collagen when subsequently treated with pepsin. Hoki nasal cartilage was prepared as described in Example 1 and glycosaminoglycans were extracted over 2 days at 8° C. using the following extraction reagents:
water
4 M urea
4 M guanidinium hydrochloride, 50 mM tris, pH 8.0
0.4 M Na₂SO₄, 10 mM Na acetate, pH 4
0.8 M MgSO₄, 10 mM Na acetate, pH 4

The glycosaminoglycan extraction was quantified as described in Example 1. The collagen was treated by limited pepsin digestion and the amount of soluble protein (minus pepsin itself) was determined.

### Results

The efficacy of the extraction reagents on GAG extraction and amount of pepsin soluble protein (atelocollagen) is shown in Table 8. Treatment with Na₂SO₄ or MgSO₄ was as effective as guanidinium HCL at extracting GAGs, and more than twice as effective as urea. Pre-treatment with Na₂SO₄ or MgSO₄ enhanced the extraction of soluble type II collagen using pepsin.

TABLE 8
GAG extraction and pepsin soluble protein
concentration from hoki cartilage.

| | GAGs | Pepsin soluble | Pepsin soluble |
|---|---|---|---|
| | extracted | protein | protein |
| Extraction | (μg/mg | concentration | extracted (μg/mg |
| reagent | cartilage) | (mg/ml) | cartilage) |
| Water | 1.02 | 0.5 | 20 |
| Urea | 106.58 | 0.7 | 28 |
| Guanidinium | 247.17 | 1.6 | 64 |
| HCL | | | |
| Na₂SO₄ | 240.69 | 1.9 | 76 |
| MgSO₄ | 236.08 | 1.6 | 64 |

1. A method for purifying type II collagen from cartilage tissue, said method comprising:
i) providing a sample of cartilage tissue;
ii) contacting the sample with a salt solution under conditions that allow at least one component of the cartilage tissue to dissolve in the salt solution, wherein the at least one component of the cartilage tissue is selected from the group consisting of proteoglycan, glycosaminoglycan, and calcium salt; and
iii) removing the salt solution and the at least one component of the cartilage tissue from the sample;
wherein step iii) removes at least about 25% of the glycosaminoglycan from the sample; and wherein, following step iii), the sample comprises type II collagen of which at least about 50% (w/w) is undenatured type II collagen.

2. The method of claim 1, wherein step iii) removes at least about 35% of the glycosaminoglycan from the sample, preferably at least about 45%.

3. The method of claim 1 wherein, following step iii), the sample comprises type II collagen of which at least about 70% (w/w) is undenatured type II collagen).

4. The method of claim 1, wherein the sample after step iii) has a solubility of less than about 80%.

5. The method of claim 1, wherein the salt solution has a pH of about 3 to about 10.

6. The method of claim 1, wherein the salt solution is a sulfate salt solution or chloride salt solution.

7. The method of claim 6, wherein the salt is sodium sulfate or magnesium sulfate.

8. The method of claim 1, wherein the salt solution has a sulfate or chloride concentration of about 0.4 M to about 2 M.

9-11. (canceled)

12. The method of claim 1, wherein sample of cartilage tissue has been obtained from:
a. a bony fish; or
b. a cartilaginous fish.

13. (canceled)

14. The method of claim 12, wherein the sample of cartilage tissue has been obtained from a cartilaginous fish, and wherein prior to step ii) the sample of cartilage tissue is subjected to a demineralisation treatment comprising contacting the sample of cartilage tissue with a solution comprising EDTA, hydrochloric acid, phosphoric acid, acetic acid, lactic acid, or any combination of any two or more of these.

15. The method of claim 1, wherein the sample of cartilage tissue has been obtained from a chicken, a domestic cow, or a sheep.

16. The method of claim 12, wherein the component dissolved by the salt solution is a glycosaminoglycan.

17. The method of claim 14, wherein the component dissolved by the salt solution is calcium salt, for example calcium phosphate.

18. The method of claim 1, wherein step iii) removes about 10% to about 100% of the proteoglycan, and/or calcium salt from the sample.

19. The method of claim 1, wherein the percentage (w/w) of type II collagen in the sample is about 10% to about 100% greater than the percentage (w/w) of type II collagen in the sample before step iii), and the percentage (w/w) of undenatured type II collagen in the sample is about 20% to about 100% greater than the percentage (w/w) of undenatured type II collagen in the sample before step iii).

20. A type II collagen composition obtained by the method of claim 1.

21. The composition of claim 20 comprising a pharmaceutically acceptable excipient.

22. The composition of claim 21 wherein the composition is a medicament or a dietary supplement.

23-24. (canceled)

25. A method for the treatment or prevention of a cartilage-related disorder, said method comprising the step of administering a dose of the composition of claim 21 to a subject in need thereof.

26. (canceled)

27. The method of claim 25, wherein the cartilage-related disorder is arthritis.
